(19) United States
(12) Patent Application Publication
(10) Pub. No.: US 2011/0184514 A1
(43) Pub. Date: Jul. 28, 2011

(54) INTRAOCULAR MENISCUS LENS PROVIDING PSEUDO-ACCOMMODATION

(76) Inventors: Robert Dimitri Angelopoulos, Fort Worth, TX (US); Michael Hamlin, Bedford, TX (US); James M. Scott, Millsap, TX (US); Costin Eugene Curatu, Crowley, TX (US)

(21) Appl. No.: 12/965,347

(22) Filed: Dec. 10, 2010

Related U.S. Application Data
(60) Provisional application No. 61/298,096, filed on Jan. 25, 2010.

(51) Int. Cl. A61F 2/16

(52) U.S. Cl. 623/6.37

(57) ABSTRACT

An intraocular lens providing pseudo-accommodation includes a haptic assembly configured to position the accommodating intraocular lens; and a meniscus-shaped optic having a convex face and a concave face. The meniscus-shaped optic has an uncompressed state within an eye when the ciliary muscles are relaxed and a compressed state within the eye when the ciliary muscles are contracted. A principal plane of the meniscus-shaped optic in the uncompressed state is anterior to the principal plane of the meniscus-shaped optic in the compressed state. A spherical aberration of the meniscus-shaped optic is substantially different in the compressed state than in the uncompressed state.

### RELATED APPLICATIONS

This application claims priority to U.S. provisional application Ser. No. 61/298,096, filed on Jan. 25, 2010, the contents which are incorporated herein by reference.

### TECHNICAL FIELD OF THE INVENTION

The present invention relates to intraocular lenses. More particularly, the present invention relates to intraocular meniscus lenses providing pseudo-accommodation.

### BACKGROUND OF THE INVENTION

The human eye is a generally spherical body defined by an outer wall called the sclera, having a transparent bulbous front portion called the cornea. The lens of the human eye is located within the generally spherical body, behind the cornea, enclosed in a capsular bag. The iris is located between the lens and the cornea, dividing the eye into an anterior chamber in front of the iris and a posterior chamber in back of the iris. A central opening in the iris, called the pupil, controls the amount of light that reaches the lens. Light is refracted by the cornea and by the lens onto the retina at the rear of the eye. The lens is a bi-convex, highly transparent structure surrounded by a thin lens capsule. The lens capsule is supported at its periphery by suspensory ligaments called zonules, which are continuous with the ciliary muscle. The focal length of the lens is changed by the ciliary muscle pulling and releasing the zonules to allow the shape of the capsular bag and the lens within to change, a process known as “accommodation.” Just in front of the zonules, between the ciliary muscle and iris, is a region referred to as the ciliary sulcus.

A cataract condition results when the material of the lens becomes clouded, thereby obstructing the passage of light. To correct this condition, three alternative forms of surgery are generally used, known as intracapsular extraction, extracapsular extraction, and phacoemulsification. In intracapsular cataract extraction, the zonules around the entire periphery of the lens capsule are severed, and the entire lens structure, including the lens capsule, is then removed. In extracapsular cataract extraction and phacoemulsification, only the clouded material within the lens capsule is removed, while the transparent posterior lens capsule wall with its peripheral portion, as well as the zonules, are left in place in the eye.

Intracapsular extraction, extracapsular extraction, and phacoemulsification eliminate the light blockage due to the cataract condition. The light entering the eye, however, is thereafter defocused due to the lack of a lens. A contact lens can be placed on the exterior surface of the eye, but this approach has the disadvantage that the patient has virtually no useful sight when the contact lens is removed. A preferred alternative is to implant an artificial lens, known as an intraocular lens (IOL), directly within the eye. An intraocular lens generally comprises a disk-shaped, transparent lens optic and two curved attachment arms referred to as haptics. The lens is implanted through an incision made near the periphery of the cornea, which may be the same incision as is used to remove the cataract. An intraocular lens may be implanted in either the anterior chamber of the eye, in front of the iris, or in the posterior chamber, behind the iris.

One drawback of using intraocular lenses is that the size and shape is typically so different from the natural crystalline lens that the accommodation process no longer works to change the focal length of the lens. This results in the lens being incapable of achieving a clear image of nearby objects, a condition known as presbyopia. Various structures have been proposed to provide some degree of pseudo-accommodation by, for example, moving the intraocular lens forward or increasing the spacing between a positive-power optic and a negative-power optic in response to contraction and relaxation of the ciliary muscles. But these devices have questionable effectiveness, particularly as the capsular bag collapses around the intraocular lens to effectively “shrink-wrap” the lens. Therefore, there remains a need for new lenses providing pseudo-accommodation, also known as “accommodating intraocular lenses.”

### SUMMARY OF THE INVENTION

An intraocular lens providing pseudo-accommodation includes a a haptic assembly configured to position the accommodating intraocular lens; and a meniscus-shaped optic having a convex face and a concave face. The meniscus-shaped optic has an uncompressed state within an eye when the ciliary muscles are relaxed and a compressed state within the eye when the ciliary muscles are contracted. A principal plane of the meniscus-shaped optic in the uncompressed state is anterior to the principal plane of the meniscus-shaped optic in the compressed state. A spherical aberration of the meniscus-shaped optic is substantially different in the compressed state than in the uncompressed state.

### BRIEF DESCRIPTION OF THE FIGURES

A more complete understanding of the present invention and the advantages thereof may be acquired by referring to the following description, taken in conjunction with the accompanying drawings in which like reference numbers indicate like features.

FIG. 1 depicts a meniscus-shaped intraocular lens (IOL) according to a particular embodiment of the present invention;

FIGS. 2A and 2B illustrate a shape change in the optic of FIG. 1 according to a particular embodiment of the present invention; and

FIGS. 3A and 3B illustrate an example wavefront showing a change in spherical aberration according to a particular embodiment of the present invention.

### DETAILED DESCRIPTION OF PREFERRED EXEMPLARY EMBODIMENTS

Various embodiments of the disclosure are illustrated in the FIGURES, like numerals being generally used to refer to like and corresponding parts of the various drawings. As used herein, the terms “comprises,” “comprising,” “includes,” “including,” “has,” “having” or any other variation thereof, are intended to cover a non-exclusive inclusion. For example, a process, article, or apparatus that comprises a list of elements is not necessarily limited to only those elements but may include other elements not expressly listed or inherent to such process, article, or apparatus. Further, unless expressly stated to the contrary, “or” refers to an inclusive or and not to an exclusive or.

Additionally, any examples or illustrations given herein are not to be regarded in any way as restrictions on, limits to, or express definitions of, any term or terms with which they are utilized. Instead, these examples or illustrations are to be regarded as being described with respect to one particular embodiment and as illustrative only. Those of ordinary skill in the art will appreciate that any term or terms with which these examples or illustrations are utilized will encompass other embodiments which may or may not be given therewith or elsewhere in the specification and all such embodiments are intended to be included within the scope of that term or terms. Language designating such nonlimiting examples and illustrations includes, but is not limited to: “for example”, “for instance”, “e.g.”, “in one embodiment”.

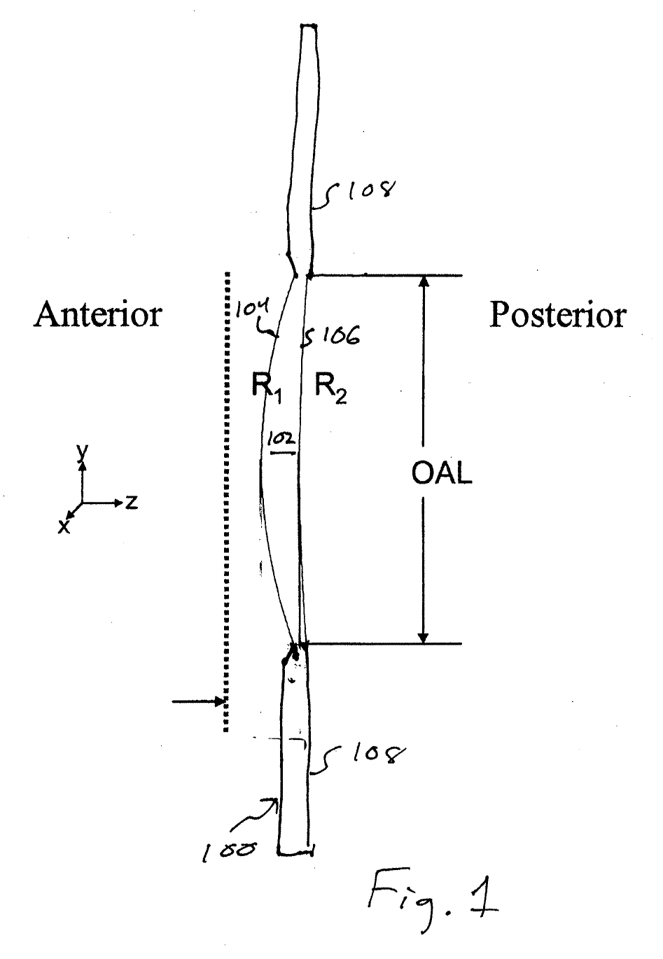

FIG. 1 depicts a meniscus-shaped intraocular lens (IOL) 100 according to a particular embodiment of the present invention. The meniscus-shaped IOL 100 has an optical portion (“optic”) 102 with an anterior convex face 104 having a radius of curvature R₁ and a posterior concave face 106 having a radius of curvature R₂. For purposes of this specification, “anterior” and “posterior” refer to the directions of the IOL 100 facing, respectively, away from and toward the retina. The “optical axis” refers to an axis extending transversely to a center of the anterior face 104 (“vertex”) in the anterior-posterior direction.

The optic 102 is formed of a generally transparent material capable of transmitting light to the retina of the eye. Any suitable material, including a wide variety of biocompatible polymeric materials, may be used. Examples of suitable materials include silicone, acrylics, hydroxyl ethyl methacrylate (HEMA), polymethyl methacrylate (PMMA) and numerous other materials known in the art. The optic 102 may also include materials for absorbing ultraviolet light, blue light, or other wavelengths to protect ocular tissue from light toxicity and/or to improve visual performance of the IOL 100.

The IOL 100 also includes a haptic assembly 108. The haptic assembly 108 fixes the position of the IOL 100 when the IOL 100 is disposed within the eye. In various embodiments of the present invention 108, the haptic assembly 108 may be configured for placement in the capsular bag or ciliary sulcus of the posterior chamber of the eye. In certain embodiments, the haptic assembly 108 could include multiple haptic arms having a proximal portion extending from the optic 102 connected by a joint to a distal portion contacting the capsular bag or ciliary sulcus. In alternative embodiments, the haptic assembly 108 could include a shaped periphery of the IOL 100 directly contacting the capsular bag or ciliary sulcus.

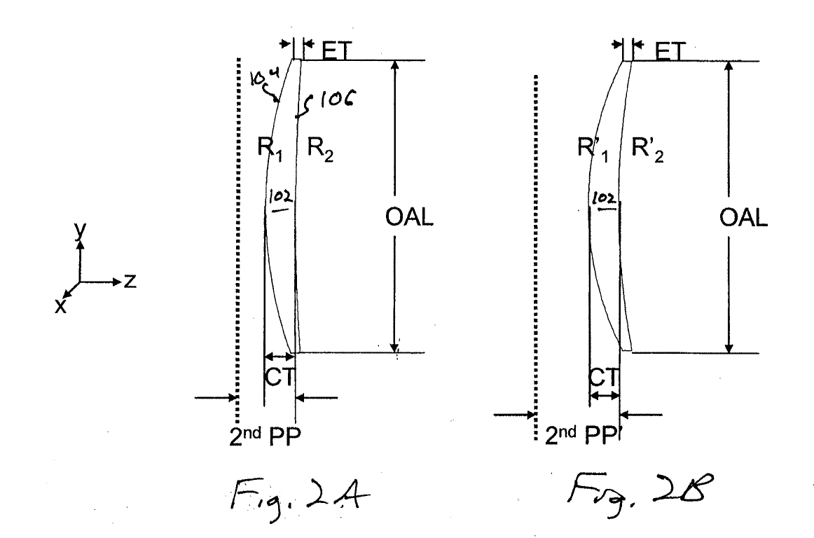

When positioned within the eye, the IOL 100 provides pseudo-accommodation by changing shape in response to contraction of the ciliary muscles. Specifically, the peripheral edge of the IOL 100 is compressed toward the optical axis so that the vertex of the anterior face 104 and the peripheral edge move relative to one another in a direction parallel to the optical axis. This compression changes the shape factor of the IOL 100 so that the principal plane is shifted posteriorly and the spherical aberration imparted by the IOL 100 substantially changes. The shape change in the optic 102 is illustrated in FIGS. 2A and 2B.

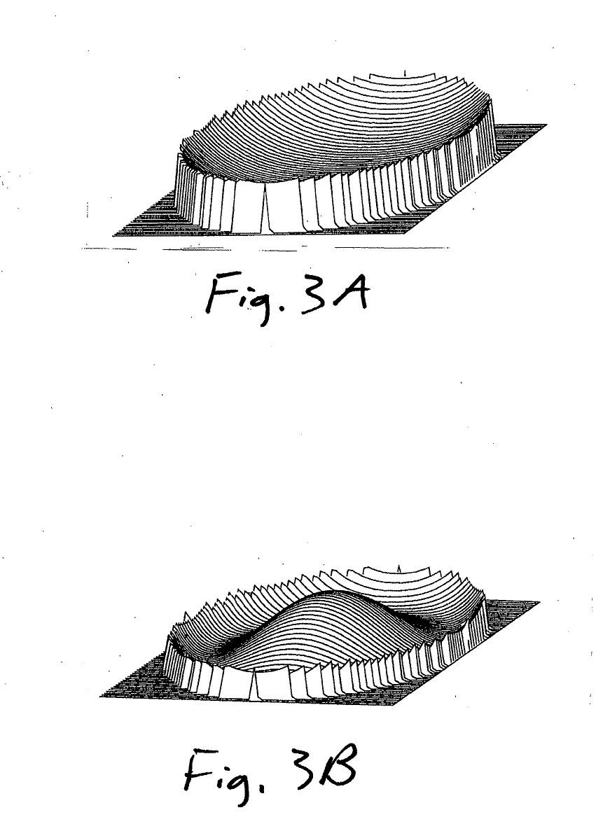

The effective change in vision can be illustrated by the wavefront at the image plane illustrated in FIGS. 3A-3B. In FIG. 3A, an example wavefront image for an IOL 100 according to a particular embodiment of the present invention is illustrated. In this example, the pupil size is set within 1 mm to 4 mm, and the wavefront is emitted from a source at infinite distance with a wavelength of 550 nm. The central peak at the image plane illustrated a sharply focused image, and the peak-to-valley spherical aberration is within 0.5 waves (about 0.135 waves RMS). FIG. 3B shows the same lens when compressed. The curvature of the wavefront illustrates the introduction of spherical aberration, with a peak-to-valley now over 3 waves (about 0.905 waves RMS). A change in object distance from an infinite distance to 140 cm corresponds to an effective power change of 0.71 D at the corneal plane or 0.92 D at the IOL plane. In general, a change of spherical aberration in the wavefront of at least 1 wave peak-to-valley for a 550-nm wavefront emitted by an object at infinity will be considered sufficient to be a substantial difference for purposes of this specification.

The mechanism to produce the shape change in the meniscus-shaped optic 102 of the IOL 100 can vary. In certain embodiments, the haptic assembly 108 can be placed in the ciliary sulcus and can transfer force from contraction of the ciliary muscles to the optic 102. In other embodiments, the haptic assembly 108 can be placed in the capsular bag so as to respond to the flattening or rounding of the capsular bag as the zonules of the eye tighten or loosen in response to relaxation and contraction of the ciliary muscles, respectively. In such embodiments, the haptic assembly 108 may be formed to exhibit a mechanical bias so that, for example, the shape change results from a spring-like response of the haptic assembly 108 to reduced tension on the capsular bag. The optic 102 can likewise exhibit a spring-like response to reduced force from the haptic assembly 108. The haptic assembly 108 may also be adapted to vault the optic in order to provide greater mechanical stability and/or more efficient mechanical response to the ciliary muscle contraction. In general, any mechanical arrangement for producing a change in shape in the optic 102 that would be contemplated by one skilled in the art may be employed in conjunction with various embodiments of the present invention.

While single-optic embodiments of the present invention have been described, it should be understood that the techniques of the present invention can be applied to multi-optic and/or multi-lens systems. Thus, for example, the meniscus-shaped IOL 100 could be placed in the ciliary sulcus anterior to a biconvex IOL in the capsular bag. In another example, a phakic IOL could be placed in the anterior chamber, and the meniscus-shaped IOL 100 could be placed in the posterior chamber. The meniscus-shaped IOL 100 may also be adapted so that the convex face 104 faces anteriorly in such combinations and, in such embodiments, the change in shape in response to contraction of the ciliary muscles can also be reversed so that the optical effect is identical. Alternatively, the meniscus-shaped IOL 100 can be adapted to provide so-called “reverse accommodation,” wherein the brain of the patient can be trained to concentrate on a distant image when the ciliary muscles are contracted and on a near image when the ciliary muscles are relaxed, thus reversing the effect of the accommodation reflex.

Although embodiments have been described in detail herein, it should be understood that the description is by way of example only and is not to be construed in a limiting sense. It is to be further understood, therefore, that numerous changes in the details of the embodiments and additional embodiments will be apparent to, and may be made by, persons of ordinary skill in the art having reference to this description. It is contemplated that all such changes and additional embodiments are within scope of the claims below and their legal equivalents.

What is claimed is:

1. An intraocular lens providing pseudo-accommodation, comprising:
a haptic assembly configured to position the accommodating intraocular lens; and
a meniscus-shaped optic comprising a convex face and a concave face, the meniscus-shaped optic having an uncompressed state within an eye when the ciliary muscles are relaxed and a compressed state within the eye when the ciliary muscles are contracted, wherein a principal plane of the meniscus-shaped optic in the uncompressed state is anterior to the principal plane of the meniscus-shaped optic in the compressed state and a spherical aberration of the meniscus-shaped optic is substantially different in the compressed state than in the uncompressed state.

2. The intraocular lens of claim 1, wherein the intraocular lens is adapted so that a vertex of the anterior convex surface remains stationary along an optical axis of the eye and the peripheral edge moves anteriorly along the optical axis when the meniscus-shaped optic is compressed into the compressed state.

3. The intraocular lens of claim 1, wherein the intraocular lens is adapted so that a vertex of the anterior convex surface moves anteriorly along an optical axis of the eye and the peripheral edge remains stationary along the optical axis.

4. The intraocular lens of claim 1, wherein a change in the spherical aberration from the compressed state to the uncompressed state for a 550-nm wavefront from infinity.

5. The intraocular lens of claim 1, wherein a change in power from the compressed state to the uncompressed state is less than 0.5 D.

6. The intraocular lens of claim 1, wherein the haptic assembly is adapted for placement in a ciliary sulcus of the eye such that the haptic assembly transfers force to the peripheral edge of the meniscus-shaped optic when the ciliary muscles contract.

7. The intraocular lens of claim 1, wherein the haptic assembly is adapted for placement in a capsular bag of the eye.

8. The intraocular lens of claim 7, wherein the haptic assembly is sized to contract the ciliary muscles of the eye such that the haptic assembly transfers force to the peripheral edge of the meniscus-shaped optic when the ciliary muscles contract.

9. The intraocular lens of claim 1, wherein an optical region of the meniscus-shaped optic is at least 4 mm in diameter.

10. The intraocular lens of claim 1, wherein the convex face is on an anterior side of the intraocular lens.
